(19) United States
(12) Patent Application Publication
(10) Pub. No.: US 2017/0112732 A1
(43) Pub. Date: Apr. 27, 2017

(54) ENCAPSULATION OF ORAL CARE ACTIVE AGENTS

(71) Applicant: KONINKLIJKE PHILIPS N.V., EINDHOVEN (NL)

(72) Inventors: FIDELINE LAURE TCHUENBOU-MAGAIA, BIRMINGHAM (GB); ZHIBING ZHANG, BIRMINGHAM (GB)

(21) Appl. No.: 15/316,907

(22) PCT Filed: Jun. 24, 2015
(86) PCT No.: PCT/EP2015/064190

§ 371 Date: Dec. 7, 2016

(30) Foreign Application Priority Data
Jun. 30, 2014 (EP) 14174980.4

(51) Int. Cl. A61K 8/11; A61K 8/31; A61Q 11/00; A61K 8/42; A61K 8/43; A61K 8/81; A61K 8/37; A61K 8/22

(52) U.S. Cl. CPC A61K 8/11; A61K 8/37; A61K 8/31; A61K 8/22; A61K 8/42; A61K 8/43; A61K 8/8111; A61Q 11/00; A61K 2800/95

(57) ABSTRACT

Disclosed is a process for encapsulating a water-soluble oral care active agent by a shell comprising a hydrophobic polymer. Two routes are described, both of which involve providing a system comprising a hydrophobic polymer, a solvent selected from the group consisting of ethyl formate, ethyl acetate, methyl acetate, propyl acetate, butyl acetate, isobutyl acetate, isopropyl acetate, isoamyl acetate, and mixtures thereof, an oral care active agent, and an oily liquid such as liquid paraffin. In the first route one first has separate phases (viz. the O/O emulsion) from which one allows precipitation by solvent removal. In the other route, the separate phases are created so as to trigger precipitation. In both routes a temperature rise takes place so as to force solvent removal, resulting in solidification.

### FIELD OF THE INVENTION

The invention pertains to a process for encapsulating a water-soluble oral care active agent. Particularly, the invention pertains to formulations for the controlled release, in the mouth, of water-soluble oral care active agents.

### BACKGROUND OF THE INVENTION

The human oral cavity, notably teeth and gums, is generally in need of oral care active agents for use in applications such as tooth whitening, tooth health, gum health and halitosis. Think of, e.g., antiplaque agents, anti-tartar agents, anti-gingivitis agents, anti-bacterial agents, bleaching agents, and others.

Such agents are generally administered from toothpastes and/or oral rinse liquids. Due to the typical environment of the oral cavity, e.g. having saliva present, a standard difficulty in the art is that active agents from toothpastes and oral rinses are quickly reducing in concentration after their application. Therefore they cannot protect the mouth for long times, and they need therefore to be applied several times daily.

In oral healthcare, increasing attention therefore goes to providing oral healthcare agents in a formulation allowing, once administered into the mouth, their release in a sustained and controlled fashion over time.

A background reference in this respect is WO 2013/093877. Herein an encapsulation system is described for the controlled release of a bleaching agent. The bleaching agent (particularly carbamide peroxide, as a source for hydrogen peroxide) is encapsulated in a shell comprising a hydrophobic material. The capsules are made by a spray cooling/congealing process. Therein paraffin wax is melted, components are added to the molten wax, and the resulting suspension is loaded into a preheated syringe, and therefrom allowed to drop on a cold surface. Whilst the method successfully results in carbamide peroxide microparticles, it would be desired to provide a more convenient process. Also, considering the water-soluble nature of oral care active agents, it were desired to provide a process that could have a more versatile in applicability to a wide range of such water-soluble oral care active agents.

Thereby it is to be considered that the agents to be encapsulated are water-soluble, and that the resulting microcapsules are used in the aqueous environment of the oral cavity. Therefore, in order to provide a process as mentioned above, it is desired to use oil-based systems, and to use hydrophobic polymers to make the microcapsules. These systems require the use of organic solvents, for which conventionally chlorinated solvents (such as methylene chloride), acetone, or ethanol are used. However, these solvents are not suitable to create a versatile encapsulation system for oral care active agents, since several important of such agents, e.g. carbamide peroxide and chlorhexidine, are soluble in these solvents. It is thus desired to provide a method for the encapsulation of water soluble oral care active agents on the basis of an oil in oil (O/O) system, yet avoiding the aforementioned solvents.

### SUMMARY OF THE INVENTION

In order to better address the foregoing desires, the invention, in one aspect, concerns a process for encapsulating a water-soluble oral care active agent by a shell comprising a hydrophobic polymer, the process comprising (a) dissolving the polymer in a solvent selected from the group consisting of ethyl formate, ethyl acetate, methyl acetate, propyl acetate, butyl acetate, isobutyl acetate, isopropyl acetate, isoamyl acetate, and mixtures thereof, so as to form a polymer solution; (b) dispersing or dissolving the oral care active agent in the polymer solution; (c) combining the polymer solution with an oily liquid so as to provide a system comprising the polymer solution and the oily liquid; raising temperature so as to extract solvent from the solution, and evaporating the solvent thereby allowing polymer particles to solidify.

In another aspect, the invention provides microparticles obtainable by the foregoing process.

In a further aspect, the invention presents the use of a solvent selected from the group consisting of ethyl formate, ethyl acetate, methyl acetate, propyl acetate, butyl acetate, isobutylacetate, isopropyl acetate, isoamyl acetate, and mixtures thereof, as a solvent for a hydrophobic polymer in making capsules comprising a water-soluble oral care active agent encapsulated in a shell of the polymer, the process comprising a step of extracting and/or evaporating the solvent thereby allowing polymer particles to solidify.

### BRIEF DESCRIPTION OF THE DRAWINGS

FIGS. 1 to 8 are all graphs showing various release profiles, as discussed in the Examples.

### DETAILED DESCRIPTION OF THE INVENTION

In a general sense, the invention is based on the judicious insight that ethyl formate, and a limited number of other esters, presents the right properties both for dissolving hydrophobic shell polymers, and for allowing phase separation to occur with the aid of an oily liquid, such as liquid paraffin, followed by evaporation of the solvent.

The system provided by the invention is particularly suitable for the encapsulation of water-soluble oral care active agents. It will be understood that a shell for the encapsulation of water-soluble oral care active agents should have a sufficiently hydrophobic character. For, if otherwise, the water-soluble nature of the oral care active agents, and the aqueous environment of the oral cavity (viz. saliva), would result in an untimely flushing away of the agents from any hydrophilic encapsulation system.

The water-soluble nature of the oral care active agents implies that, in combination with a hydrophobic shell polymer, the agents will inevitably form a biphasic system. This, in turn, presents a challenge for encapsulation, as a system needs to be provided that results in the formation of a shell around the actives, rather than just a mixture of actives and polymer. Whilst several methods exist in the art such as oil-in-oil (O/O) emulsion solvent evaporation and phase separation or coacervation, a challenge lies in finding the right system specifically suited for oral care active agents. The present invention allows the formation of encapsulates with the desired properties, including particles size below 300 μm and preferably below 100 μm that allow a sustained release of water soluble actives in a controlled manner in the oral cavity for a substantial period of time (from minutes to days depending on the formulation and the type of actives).

The polymer, including copolymers and combinations of polymers, used in the capsules of the invention is hydrophobic. Hydrophobic polymers generally have relatively long-term maintenance of their structural properties in contact with water or saliva in the oral cavity when compared to hydrophilic polymer which rapidly become water swollen and dissolve in the oral cavity.

An interesting polymer to be used as a carrier material in the invention, is, e.g., hydrophobic cellulose derivatives such as ethyl cellulose and cellulose acetate butyrate (CAB). CAB and ethyl celulose can be obtained, e.g., from Sigma Aldrich and Eastman Chemical. Another interesting type of polymer is a series if copolymers of ethyl acetate and methyl methacrylate, further having a low content of methacrylic acid ester with quaternary ammonium groups. This type of copolymer is known, e.g., as Eudragit RS (in various grades) produced by Evonik. Yet another interesting polymer is poly(methyl methacrylate) and polyesters such as polycaprolactone (PCL) which are widely obtainable. Also mixtures of polymers can be used.

Suitable oily liquids are not only liquid paraffin, but in any liquid, or mixture, that is miscible with the solvent and which does not dissolve the polymer, such as heptane. A suitable ratio of ethyl formate:heptane is 1:5 to 1:15, preferably 1:10. Other suitable oily liquids include, e.g., mineral oil dodecane, hexadecane, methyl myristate, methyl laurate, and mixtures thereof.

Oral care active agents are administered into the oral cavity, in order to exert their action on teeth, gums, or on other surfaces within the mouth. The oral care active agents can be whitening agents, antiplaque agents, anti-tartar agents, anti-gingivitis agents, anti-bacterial agents, and combinations thereof. These agents are known to the skilled person, and the invention does not depend on any specific agent. Rather, the invention provides a versatile process to encapsulate a wide range of different such active agents. Particularly interesting agents are bleaching agents such as hydrogen peroxide and sodium carbonate peroxide, or hydrogen peroxide precursors, e.g. carbamide peroxide. Also of particular interest are antibacterial agents such as chlorhexidine or other bactericides, such as triclosan, and/or bacteriostatic agents such as zinc chloride or zinc citrate. Further interesting agents that can be encapsulated in accordance with the invention are anti-tartar agents, e.g., pyrophosphates such as tetrasodium pyrophosphate (TSPP). Also of interest for encapsulation is fluoride, which is usually present in dentifrices and mouthwashes by virtue of its ability to prevent cavities. Suitable fluorides are, e.g., sodium fluoride or sodium mono fluorophosphate.

The active loading preferably ranges from 5% to 50%, more preferably 8% to 33%, with a ratio active: carrier in the formulation of preferably 1:6 to 1:1, more preferably 1:4 to 1:2.

The invention can be realized generally according to two routes. In a first route, the polymer is dissolved in the solvent so as to make a polymer solution. Thereupon, one or more active agents to be encapsulated are added to the polymer solution. Next, the polymer solution is emulsified into the oily liquid, so as to form an emulsion comprising active agent(s), hydrophobic polymer, and oily liquid. Hereby one can add an emulsifier to lower the surface tension between the two phases and to prepare particles with smaller size. However it is also possible to prepare microparticles without using an emulsifier.

Then the temperature of the emulsion is raised. Typically, the initial temperature is within a range of higher than 0° C. to room temperature, such as 18° C., preferably from 5° C.-10° C. The temperature is than typically raised to above room temperature, preferably of from 20° C. to 35° C. This causes the solvent to be extracted and evaporated, which results in particle precipitation and solidification.

In a second route, one dissolves the polymer in the solvent to make a polymer solution, and adds one or more oral care active agents to the polymer solution just as in the first route. Rather than making an emulsion as above, in the second route the oily liquid is added without an emulsifying step. Thereby the oily liquid, particularly liquid paraffin, acts as an anti-solvent. This results in particle precipitation. Thereafter the temperature is raised so as to extract and evaporate solvent, thereby solidifying the formed particles. Thereby the initial temperature is typically well below room temperature, e.g. up to 10° C., and the temperature is raised to 10° C.-35° C. The second route is of additional interest in the event that solvents are used with relatively higher boiling points. In that case the solidification of microparticles can be achieved by increasing the quantity of the anti-solvent, as compared to a solvent having a relatively low boiling point, such as ethyl formate or ethyl acetate. In this embodiment, another step may include adding a hardening solvent, such as hexane, to coacervate droplets formed, as to aid in the removal of the solvent, and thereby solidify the coacervate droplets.

Also, combination of higher and lower boiling solvents can be used in either route.

Without wishing to be bound by theory, the inventors believe that the foregoing routes differ in terms of the mechanism of particles formation. In the emulsion route, droplets of polymer containing the actives is formed in the emulsion and these are then hardened as the organic solvent diffuses/evaporates. When using an anti-solvent, microparticles are formed because of the separation of a liquid phase from the liquid phase containing the shell material which results in reduction of the solubility of the shell material and its precipitation thereby encapsulating the core material.

Hence, the invention in both of the aforementioned routes involves providing a system comprising a hydrophobic polymer, a solvent selected from the group consisting of ethyl formate, ethyl acetate, methyl acetate, propyl acetate, butyl acetate, isobutylacetate, isopropyl acetate, isoamyl acetate, and mixtures thereof, an oral care active agent, and an oily liquid such as liquid paraffin. In the first route one first has separate phases (viz. the O/O emulsion) from which one allows precipitation by solvent removal. In the other route, the separate phases are created so as to trigger precipitation. In both routes a temperature rise takes place so as to force solvent removal, resulting in solidification.

In a further embodiment, swellable or gelling materials can be added to the formulation. This is of particular interest in the event that the capsules of the invention are mixed into a varnish formulation such as used in the whitening of teeth.

Further ingredients customary in oral health care can be added, e.g., precursors of amorphous calcium phosphate, which are capable of acting as de-sensitizing agents, particularly in connection with dental bleaching. Other de-sensitizing agents may also be used. Suitable desensitizing agents, if added, may include, for example, alkali nitrates such as potassium nitrate, sodium nitrate and lithium nitrate; and other potassium salts such as potassium chloride and potassium bicarbonate.

Further agents can be, e.g., antiplaque agents, anti-tartar agents, anti-gingivitis agents, anti-bacterial agents, anti-caries agents, and combinations thereof.

A preferred anti-caries agent is fluoride. The fluoride can be provided as a separate component, but preferably is comprised in either the calcium component or the phosphor component. Suitable fluoride sources include sodium fluoride, stannous fluoride, sodium monofluorophosphate, zinc ammonium fluoride, tin ammonium fluoride, calcium fluoride, cobalt ammonium fluoride potassium fluoride, lithium fluoride, ammonium fluoride, zinc ammonium fluoride, tin ammonium fluoride, calcium fluoride, cobalt ammonium fluoride, water soluble amine hydrofluorides, or mixtures thereof. The fluoride-containing component preferably comprises the fluoride source in an amount of at least 0.001%, more preferably, from 0.01 to 12%, and most preferably, from 0.1 to 5% by weight of the total system.

Other possible oral healthcare active agents that can be included in either or both of the components are, e.g., antibacterial agents. These include, for example, phenolics and salicylamides, and sources of certain metal ions such as zinc, copper, silver and stannous ions, for example in salt form such as zinc, copper and stannous chloride, and silver nitrate. These are present in art-known small quantities when used. In addition, optional additives can be present in either or both of the components. These include, e.g., humectants, flavourings, colouring agents, anti-plaque agents, anti-staining compounds, pH adjusting agents, excipients such as emollients, preservatives, other types of stabilizers such as antioxidants, chelating agents, tonicity modifiers (such as sodium chloride, mannitol, sorbitol or glucose), spreading agents, and water soluble lubricants, such as propylene glycol, glycerol or polyethylene glycol. The concentration of each may easily be determined by a person skilled in the art.

Humectants include water, polyols, such as glycerol, sorbitol, polyethylene glycols, propylene glycols, hydrogenated partially hydrolysed polysaccharides and the like. A single humectant or a combination is also contemplated. They are generally present in amounts of, for example, up to about 85%, more for example, from about 15% to about 75% of the formulation.

In the various embodiments of the methods according to the invention, anti-aggregating agents such as magnesium stearate (MS), calcium stearate or other particles can be added. Particularly, in the invention it is possible to add such agents to either the dispersed phase or, unconventionally, the continuous phase. The inventors surprisingly found that the release profile of the oral care active agent can be tuned just by changing the way MS is added into the system. Without wishing to be bound by theory, the inventors hypothesise that microparticles with a more compact structure were formed when MS was dispersed in the continuous phase prior to emulsion formation.

Further, the capsules made according to the invention can be coated, as desired with one or more further layers of release-controlling materials. A particularly interesting polymer to be used as a coating, are hydrophobic polymers, which are soluble in heptane and which can be used as an anti-solvent for the esters, and particularly for ethyl formate. A preferred hydrophobic polymer is polyisobutylene. Polyisobutylene is known from the manufacture of pressure-sensitive adhesives and sealants or pressure-sensitive adhesives for transdermal drug delivery systems. It is approved by the FDA (the US regulatory agency for food and drugs) for chewing gum and medical device production, is highly resistant to oxidation and has a very low permeability to small molecule (e.g. methanol, moisture and gas). The inventors have surprisingly found that polyisobutylene is well suitable as a release-controlling coating on the capsules according to the invention.

In another aspect, the invention provides microparticles obtainable by the foregoing process. These microparticles can be recognized with reference to remaining (residual) solvent or oily liquid. The residual solvent is discussed with reference to ethyl formate, but is similarly applicable to the other esters or mixtures of esters.

The concentrations of the solvent or the oil liquid in microparticles may be monitored by collecting a given quantity of microparticles (at different stages of the production, up to and including the final product), then extract the solvent and the oily liquid from the microparticles with appropriate solvents such as dimethyl sulfoxide, dimethyl formamide, ethanol or non polar solvents such as petroleum ether and analyse using a gas chromatograph equipped with a FID or gas chromatography coupled with mass spectrometry.

In order to eliminate the oily liquid from the produced microparticles, these were washed with solvents in which the oily liquid is soluble and vacuum dried. These solvents include hexane, heptane and petroleum ether. Quantitative analysis of the residual solvents in the final microparticles can be performed as described in the European Pharmacopoeia “Identification and Control of Residual Solvents (2.4.24.), 2010, European Pharmacopoeia (6th ed.) European Department for the Quality of Medicines, Strasbourg”.

The residual ethyl formate in the microparticles after drying was determined as follows: 10 mg of free flowing microparticles were crushed using a mortar and pestle and mixed with 3 mL HPLC grade ethanol and the mixture was transferred into a glass vial (20 mL). The mortar and pestle were rinsed three times with 3 mL ethanol and to make the total extraction solution up to 10 mL. The vial was then closed with butyl rubber stopper and aluminium crimp seal and sealed with a hand crimper before mixing for 2 h with a rotating laboratory mixer. The sample solution was filtered with a 0.22 μm syringe filter and the content of ethyl formate determined by a gas chromatograph equipped with a flame ionisation detector and Helium as a carrier gas

The concentrations of ethyl formate in unknown samples were calculated based on a calibration curve constructed by integrating peak areas of ethyl formate standards of known concentrations obtained with different concentration of ethyl formate diluted with anhydrous ethanol.

TABLE 1
Residual solvent in microparticles

| | | Residual |
|---|---|---|
| | | Ethyl |
| | | formate |
| Sample | Description | (PPm) |
| 1 | CAB microparticles without active prepared with | 10. ± 1.2 |
| | MS in the dispersed phase | |
| 2 | CAB microparticles prepared as “1” loaded with | 11.9 ± 2.7 |
| | NaF; CAB:NaF, 4:1 | |
| 3 | CAB microparticle prepared as “1” loaded with | 10.2 ± 3.2 |
| | NaF; CAB:NaF, 4:2 | |
| 4 | CAB microparticles without active prepared with | 13.2 ± 0.7 |
| | MS in the continuous phase | |
| 5 | CAB microparticles prepared as “1” loaded with | 9.3 ± 0.2 |
| | carbamide peroxide (CP); CAB:CP, 4:2 | |
| 6 | Eudragit EPO microparticles without active prepared | 10.2 ± 0.5 |
| | microparticle prepared as “1” | |
| 7 | Eudragit RS-PEO (1:1) microparticles without active | 12.0 ± 0.4 |
| | microparticle prepared as “1” | |
| 8 | Eudragit RS microparticle prepared as “1” loaded | 9.2 ± 0.1 |
| | with carbamide peroxide (CP); RS:CP, 4:2 | |

Mean ± S.D;
n = 3.

In a further aspect, the invention also concerns the use of a solvent as defined hereinbefore as a solvent for a hydrophobic polymer in making capsules comprising a water-soluble oral care active agent encapsulated in a shell of the polymer. The solvent is thereby selected from the group consisting of ethyl formate, ethyl acetate, methyl acetate, propyl acetate, butyl acetate, isobutylacetate, isopropyl acetate, isoamyl acetate, and mixtures thereof, and preferably is ethyl formate or, more preferably ethyl acetate. In putting to practice this novel use of these solvents, the method of making the capsules is preferably process in accordance with one or more of the embodiments of the process for encapsulating a water-soluble oral care active agent as described hereinbefore. In this use, the process comprises a step of evaporating the solvent thereby allowing polymer particles to solidify, particularly in either of the two routes described above.

While the invention has been illustrated and described in detail in the drawings and foregoing description, such illustration and description are to be considered illustrative or exemplary and not restrictive; the invention is not limited to the disclosed embodiments.

For example, it is possible to operate the invention in an embodiment wherein part of the water-soluble oral care active agent is encapsulated via an emulsifying step, and another part via allowing the oily liquid to act as an anti-solvent.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain features of the invention are recited in mutually different dependent claims does not indicate that a combination of these features cannot be used to advantage.

In sum, we hereby disclose a process for encapsulating a water-soluble oral care active agent by a shell comprising a hydrophobic polymer. Two routes are described, both of which involve providing a system comprising a hydrophobic polymer, a solvent selected from the group consisting of ethyl formate, ethyl acetate, methyl acetate, propyl acetate, butyl acetate, isobutyl acetate, isopropyl acetate, isoamyl acetate, and mixtures thereof, an oral care active agent, and an oily liquid such as liquid paraffin. In the first route one first has separate phases (viz. the O/O emulsion) from which one allows precipitation by solvent removal. In the other route, the separate phases are created so as to trigger precipitation. In both routes a temperature rise takes place so as to force solvent removal, resulting in solidification.

The invention will be further explained hereinafter with reference to the examples and figures. These illustrate the invention, but do not limit it.

### EXAMPLE 1

A polymer solution was made by dissolving 6 g of cellulose acetate overnight (14 h) in 40 ml of ethyl formate in a glass sealed container at room temperature. Chlorhexidine diacetate (1.5 g) was then dispersed into the pre-cooled polymer solution, which was placed on ice while stirring with magnetic stirrer at 300 rpm for 10 minutes before pouring the suspension into a jacketed reactor connected to a circulated water bath to control the temperature at 5° C. The active/polymer ratio based on weight was 1/4. The anti-solvent, liquid paraffin (200 mL) containing magnesium stearate, anti-aggregating and release rate controlling agent (MS, 1 g) and polyglycerol polyricinoleate, emulsifier (PGPR, 0.5 g) prepared by blending the mixture with a high shear rotor stator mixer (Silverson L4RT) at 8000 rpm for 2 min while cooling the system with an ice bath was pumped (6 mL/min) into the active-polymer suspension previously prepared whilst mixing with a pitched blade impeller fitted to an IKA EUROSTAR overhead stirrer at 600 rpm, and to promote polymer precipitation around the active. The reactor temperature was then raised to 20° C. and the system left stirred for 2 hours at 600 RPM to allow further extraction and evaporation of ethyl formate, resulting in microparticles solidification.

This method is referred to herein as the standard phase separation method (PS). Microparticles were also made via a modified phase separation method (MPS) wherein liquid paraffin was divided into 2 parts: the first part, 80 mL, containing PGPR with or without MS and other part (120 mL) consisting of pure liquid paraffin. The pre-cooled polymer solution containing active was loaded into a syringe and added into the reactor containing the first part of anti-solvent saturated with 15% (v/v) ethyl formate (12 mL) at 5° C. This percentage of ethyl formate was chosen based on the calculated solubility of the solvent in liquid paraffin (Table 3). The mixture was stirred with a pitched blade for 20 min at 600 rpm before pumping the remaining 120 mL pure liquid paraffin into the reactor. The temperature of the reactor was then raised to 20° C. as described above and the resulting microparticles were separated from the liquid paraffin by centrifugation at 3000 rpm for 4 min. The oily microparticles were loaded into a Duran® sintered disc filter funnel, washed three times with cold n-hexane and vacuum dried overnight. A free flowing powder of microparticles was obtained.

To determine the release profile of chlorhexidine, 50 mg of microparticles was loaded in a tea bag (t-sac, GmbH, Hannover, Germany) and placed in 250 ml bottle. 100 mL of deionised water preconditioned at 37° C. were added to the bottle and placed in an orbital shaker (HT INFORS) at 150 rpm, 37° C. 5 mL of the liquid was withdrawn at a predetermined times and replaced with an equivalent volume of the fresh medium maintained at 37° C. and the quantity for chlorhexidine present in the aliquot determined using a spectrophotometer at 255 nm with reference to the standard curve.

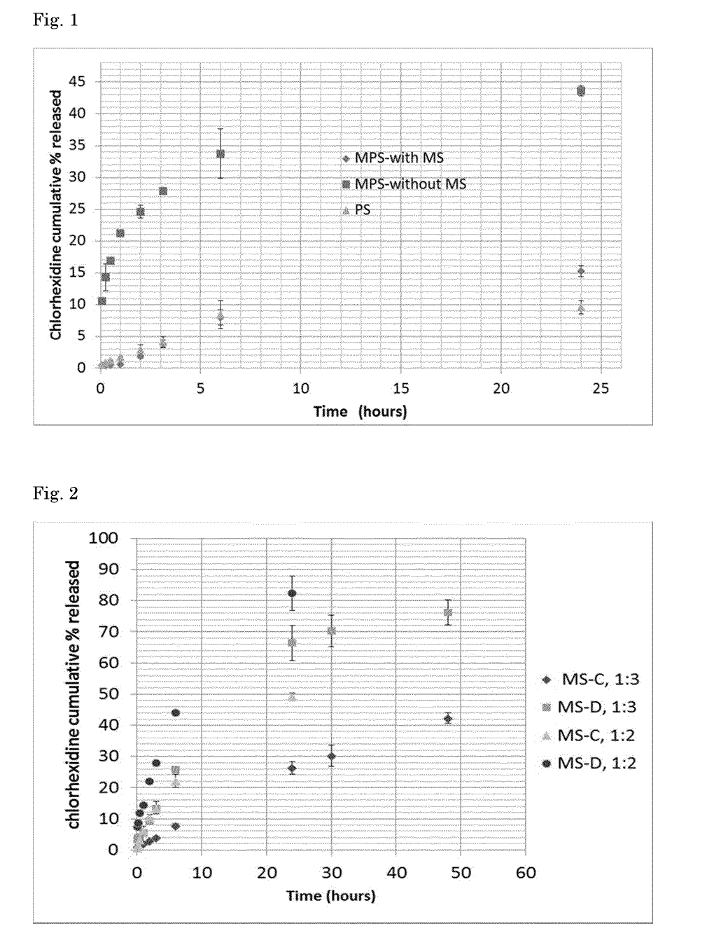

FIG. 1 is a graph showing the effect of the production methods and magnesium stearate on the release profile of Chlorhexidine. PS refers to phase separation with MS (magnesium stearate) in the formulation. MPS-with MS and MPS-without MS correspond to CAB microparticles made via a modified phase separation. The pumping rate of the anti-solvent was 3 mL/min for MPS method and 6 mL/min for PS method as the lower pumping rate resulted in polymer agglomeration due to the increase in the viscosity of the system as ethyl formate prematurely evaporated before enough of the anti-solvent was added into the reactor.

### Determination of Ethyl Formate Solubility in Paraffin Oil.

12 mL of ethyl formate (0.917 g/cm³) were added to 18 mL of liquid paraffin (density, 0.830 g/cm³) in a glass bottle. The bottle was then closed with butyl rubber stopper and aluminium crimp seal and seal with a hand crimper before vigorously mixing the system with a vortex for 2 min. The bottle containing the mixture was immersed in a temperature-controlled jacketed beaker for 24 h and to allow the formation of two distinct layers, an upper layer consisting of ethyl formate and Liquid paraffin and the lower layer with only ethyl formate. Samples were withdrawn from the upper layer with a needle, filtered and analysed using a gas chromatography with flame ionisation detector (GC-FID) under the conditions presented in table 2. The quantity of ethyl formate in the mixture was inferred from the calibration curve obtained with different concentration of ethyl formate diluted with anhydrous ethanol.

TABLE 2
Operation conditions for the quantification of ethyl formate by GC-FID

| Parameters | Set values |
|---|---|
| Carrier gas type | Helium |
| Carrier gas flow rate | 1.5 ml/min |
| Detector gas | 30 ml/min |
| Sample loop volume | Auto injection by using auto sampler |
| Sample size injected | 0.2 μl |
| Column | DB-Wax: |
| | capillary column, 0.25 mm ID and 0.25 μm |
| | film thickness, 30 m length |
| Injector temperature | 180° C. |
| Oven temperature | Initial oven temperature was 35° C., held for |
| | 5 minutes, then 10° C./min up to 230° C. |
| Detector temperature | FID@ 250° C. |

TABLE 3
Solubility of ethyl formate in liquid paraffin at different temperatures

Temperature (° C.)

| | 5 | 10 | 20 | 30 |
|---|---|---|---|---|
| Ethyl formate solubility in | 13.4 | 16.3 | 26.8 | 38.9 |
| liquid paraffin (% v/v) | | | | |

### EXAMPLE 2

4 g of Cellulose acetate was dissolved in 20 ml of Ethyl Formate. The continuous phase was prepared by adding 0.85 g of MS to 70 ml of Liquid Paraffin containing 0.35 g of PGPR emulsifier and mixed for 2 min with a high shear rotor stator mixer (Silverson L4RT) at 8000 rpm) while cooling the system with an ice bath. A given quantity of chlorhexidine was then added to the polymer solution, placed on ice, to reach a specific active/polymer ration (1/2; 1/3 while stirring. The mixture was further stirred with a magnetic stirrer at 300 rpm for 10 minutes before it was added into the reactor containing the previously prepared continuous phase, saturated with 30% (v/v) ethyl formate (i.e. 21 mL) and cooled to 5° C. The mixture was magnetically agitated at 400 rpm using a six-pitched Rushton impeller for 2 hours to create the emulsion. The oil in oil emulsion was gradually heated to 20° C., stirred at this temperature for 16 h to allow the diffusion of ethyl formate into liquid paraffin and its evaporation through the air/liquid interface leading to microparticles solidification. Microparticles were separated from the liquid paraffin by centrifuging at 3000 rpm for 4 min, washed three times with cold n-hexane and vacuum dried overnight. When MS was added into the dispersed, cellulose acetate butyrate was dissolved in 15 ml ethyl formate and the other 5 mL was used to dispersed MS (0.85 g) using an ultrasonic bath containing ice for 30 min. The emulsion and microparticles were then obtained as in the case where MS was in the continuous phase.

FIG. 2 is a graph showing the effect of magnesium stearate (MS) in continuous or dispersed phase upon chlorhexidine release profile. MS-D and MS-C correspond to magnesium stearate in dispersed and continuous phase, respectively. 1:3 and 1:2 are active/polymer ratio of 1:3 and 1:2, respectively.

### EXAMPLE 3

NaF loaded microparticles were prepared by emulsion solvent evaporation according to a two-level factorial design (Table 4) with three factors, polymer concentration (10% and 20% w/v) solvent evaporation temperature (10° C. and 20° C.) and solvent concentration in the continuous phase (i.e. the quantity of ethyl formate in the liquid paraffin at the start of the emulsification, 15% (10.5 mL) and 30% (21 mL) v/v). Samples were made according to the procedure described in Example 2 with MS in the dispersed phase. 0.85 g magnesium stearate dispersed in 5 mL ethyl formate with an ultrasonic bath containing ice for 30 min was added into the CAB solution containing 1 g or 0.5 g of NaF to reach active polymer ratio of 1:4. The poly solution was prepared by dissolving 2 g and 4 g of the polymer in 15 mL ethyl formate for 10% and 20% polymer in total ethyl formate, respectively. NaF was dispersed into the polymer solution for 7 min before adding MS and the mixture was further stirred for 3 min using magnetic stirrer and to form the dispersed phase. At the same time, the continuous phase made of 0.35 g PGPR and 70 mL liquid paraffin was transferred into the reactor at 5° C. and 10.5 mL or 21 mL of cold ethyl formate was added to the system to reach a concentration of 15% or 30% v/v. The dispersed phase was then added into the continuous phase and stirred at 600 rpm for two hours at temperature of 5° C. to produce an O/O emulsion followed by an increase of the temperature to 20° C. for microparticles hardening as in Example 2. Sample was also prepared in the same conditions but with a ratio NaF:CAB of 1:2 (2 g NaF, sample 9).

The encapsulation efficiency (EE) and the loading efficiency (LE) were calculated according to equations Eq. 1 and 2 after extraction analysis of the encapsulated NaF from the microparticles. For this, 50 mL of deionised water were added to 25 mg of NaF loaded microparticles crushed in a glass mortar with a pestle. The mixture was stirred with the pestle from time to time (4 times) to extract the active for one hour followed by filtration of the extract with a hydrophilic Polyethersulfone filter unit (Millex-GP, 0.22 μm) and discarding of the first 15 ml. 4.4 mL of the filtrate was mixed with to 4.4 mL TISAB III buffer and the concentration of fluoride determined using fluoride selective electrode meter (Eutech Instruments PTE LTD) with reference to a standard curve.

The particle size of the microparticles was determined with a particle size analyser, (Mastersizer Hydro 2000SM, Malvern Instrument Ltd, UK).

$$\begin{matrix}EE ( \% ) = \frac{Actual active content}{Theoritical active content} \times 100 & Eq . 1 \\ LE ( \% ) = \frac{Mass of active in microspheres}{Mass of microspheres} \times 100 & Eq . 2\end{matrix}$$

TABLE 4
Influence of polymer concentration, solvent volume in continuous
phase and solvent evaporation temperature on encapsulation
efficiency and particle size of CAB microparticles loaded with NaF.

| | | Solvent in | | | | Surface |
|---|---|---|---|---|---|---|
| | CAB | Cont. | Evap. | NaF | | weighted |
| | Conc. | Phase | Temp. | Loading | EE | diameter |
| # | (% w/v) | (% v/v) | (° C.) | (% w/w) | (% w/w) | d(3,2) (μm) |
| 1 | 20 | 30 | 20 | 18 ± 1 | 90 ± 1 | 100 ± 1 |
| 2 | 20 | 30 | 10 | 16 ± 1 | 79 ± 6 | 146 ± 20 |
| 3 | 10 | 30 | 20 | 15 ± 1 | 72 ± 2 | 43 ± 3 |
| 4 | 20 | 15 | 10 | 16 ± 1 | 79 ± 2 | 286 ± 3 |
| 5. | 10 | 30 | 10 | 13 ± 1 | 64 ± 1 | 137 ± 16 |
| 6 | 10 | 15 | 10 | 14 ± 1 | 70 ± 1 | 132 ± 19 |
| 7 | 10 | 15 | 20 | 15 ± 1 | 74 ± 1 | 48 ± 5 |
| 8 | 20 | 15 | 20 | 17 ± 1 | 87 ± 1 | 149 ± 22 |
| 9 | 20 | 30 | 20 | 33 ± 2 | 98 ± 5 | 270 ± 1 |

Microparticles were made via O/O emulsion solvent evaporation with active/polymer ratio 1/4 and the impeller speed 600 rpm. MS was added into the dispersed phase.

The release studies of NaF microparticles was carried out at 37° C. for 24 hours or a week. 50 mg of microparticles was loaded in a tea bag (t-sac, GmbH, Hannover, Germany) and placed in 250 ml bottle. 100 mL of deionised water preconditioned at 37° C. were added into the bottle and placed in an orbital shaker (HT INFORS) at 150 rpm, 37° C. 5 mL of the release medium were withdrawn at a predetermined time and replaced by 5 mL of deionised water and the quantity of fluoride present in the aliquot determined as described above with 2.2 ml sample diluted with 2.2 ml deionised water and 4.4 ml TISAB III buffer solution.

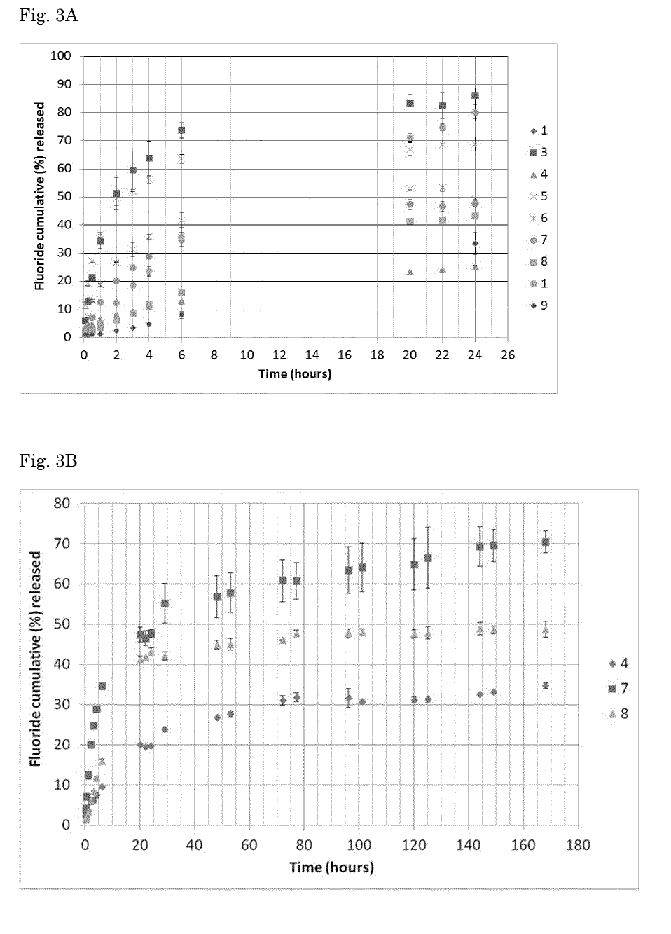

FIG. 3A is a graph showing the release of fluoride over 24 h, with reference to the influence of polymer concentration, solvent volume in continuous phase and solvent evaporation temperature upon the release profile of fluoride.

FIG. 3B is a similar graph, showing the release of fluoride over a week.

### EXAMPLE 4

Zinc diacetate (ZnAc) loaded microparticles were prepared via modified phase separation (sample Z18) or emulsion solvent evaporation (samples Z19 and Z21) or (Z18). For Z18, 6 g of CAB was dissolved in ethyl formate for 14 h (overnight) and 1.5 g of ZnAc was dispersed into the polymer solution for 3 min followed by the addition of 1 g of MS suspension in 5 mL ethyl formate. The mixture was further stirred with at magnetic bar at 300 rpm on ice before pouring into a cold reactor containing 40 mL of liquid paraffin, 0.5 g PGPR and 6 mL of ethyl formate. The whole system was blended at 5° C. for 20 min, then 160 mL liquid paraffin was pumped into the reactor (pumping rate 6 mL/min) and the reactor temperature was raised to 20° C. and stirred for 2 h to allow solvent extraction/evaporation and formation of microparticles as in Example 1. Z19 and Z21 were made by dispersing 1 g and 1.5 g of ZnAc into CAB solution prepared by dissolving 3 g of CAB into 20 mL ethyl formate for 3 minutes. A suspension of 0.85 g MS in 5 mL ethyl formate then added into the mixture and stirred for further two minutes. The mixture was poured into 70 mL liquid paraffin containing 0.35 g PGPR and 15 mL of ethyl formate and stirred with a at 600 rpm 5° C., for 2 h to make an O/O emulsion. The temperature of the reactor was raised to 20° C., 15 h to allow the evaporation of the solvent and solidification of microparticles. The free flowing microparticles loaded with ZnAc were then obtained as in Example 3.

A release study of zinc loaded-microparticles was performed in the same conditions as for chlorhexidine and fluoride and zinc analysed with an atomic absorption spectrometer (AAnalyst 200, PerkinElmer) after suitably diluting with the release medium.

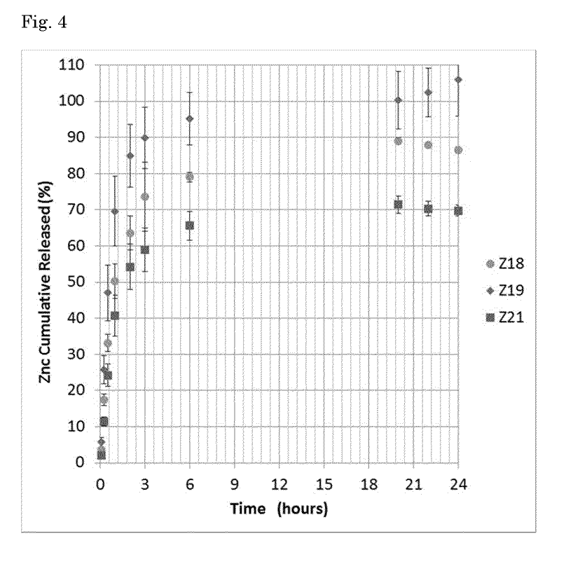

FIG. 4 is a graph showing the release profile of Zinc from CAB microparticles prepared via MPS and emulsion solvent evaporation.

### EXAMPLE 5

Chlorhexidine loaded polycaprolactone microparticles were made as described in Example 2 with MS in the continuous phase. 1 g of chlorhexidine was dispersed in polycaprolactone (PCL) solution prepared by dissolving 4 g of PCL in 15 ml of Ethyl formate. The continuous phase was made of 70 ml of liquid paraffin, 0.35 g PGPR surfactant and 0.55 g MS, 0.25 MS or no MS blended with a high shear mixer (Silverson L4RT). The emulsion and free flowing microparticles loaded with chlorhexidine diacetate were then obtained as in Example 2.

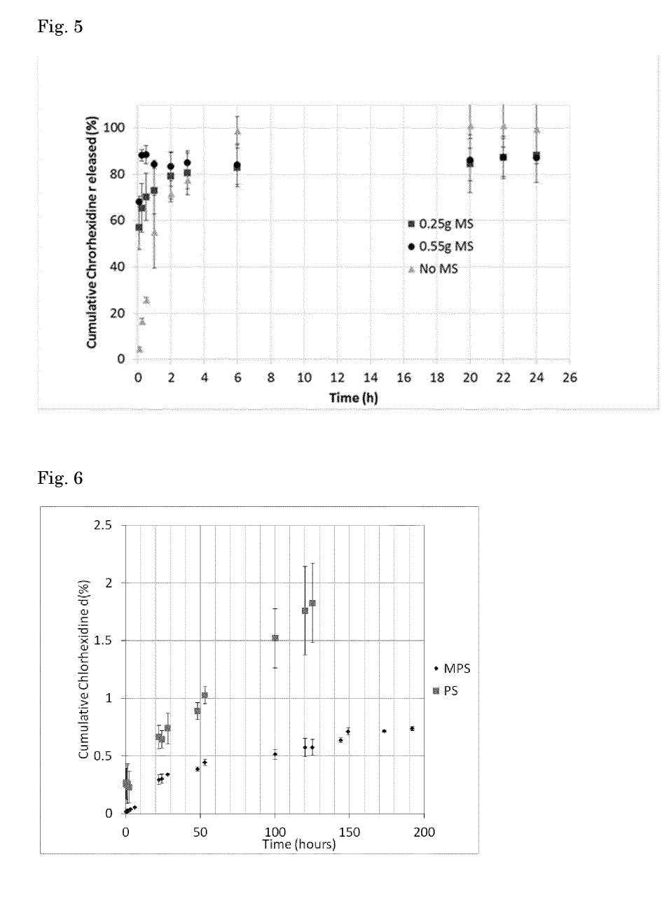

FIG. 5 is a graph showing the release profile of chlorhexidine released from PCL microparticles.

### EXAMPLE 6

CAB-microparticles loaded with chlorhexidine were prepared via solvent evaporation and modified solvent evaporation as described in the Example 1.

1.5 g or 3 g of chlorhexidine was dispersed into CAB solution prepared by dissolving 6 g CAB in 40 mL ethyl formate for 14 h (overnight) and to yield a ratio active/polymer of 1/4 and 1/2, respectively. PIB was first dissolved into liquid paraffin (200 mL) at about 70° C. before cooling to the working temperature. MS, 0.25 g was added to this liquid paraffin and the mixture was blended with a high shear rotor stator mixer (Silverson L4RT) at 8000 rpm for 2 min while cooling the system with an ice bath before pumping (6 mL/min) into the active-polymer suspension (1/4) for the preparation of microparticles as in Example 1. Also 40 mL of liquid paraffin containing 0.25 g MS was blended with a Silverson as above and mixed with a suspension of active-CAB (1/2) for 20 min in a cold reactor after being saturated with 6 mL of ethyl formate as for the standard modified phase separation. 160 mL of liquid paraffin containing 0.25 g MS was then pumped (3 mL/min) into the reactor temperature and the reactor temperature was raised to 20° C. and stirred for 2 h to allow solvent extraction/evaporation and formation of microparticles as in Example 1.

FIG. 6 is a graph showing the release profile over a week of CAB microparticles loaded with chlorhexidine made via phase separation (PS) or modified phase separation method (MPS) in the presence of PIB dissolve in the anti-solvent. The active: CAB ratio was 1/4 and 1/2 for PS and MPS, respectively.

### EXAMPLE 7

Carbamide peroxide (CP) loaded-microparticles were made via emulsion solvent evaporation using acetone or ethyl formate as solvent and Eudragit RS as a carrier as described in Example 3 according to the formulations presented in Table 4. Precirol ATO 5 (Glycerol distearate) or MS were used as anti-aggregating agents and no solvent was added into liquid paraffin when acetone was used as solvent.

For the encapsulation efficiency, 25 ml of HPLC grade pre-cooled ethanol were added into 25 mg of sample crushed with a pestle in a pre-cooled mortar placed on ice. The mixture was stirred with the pestle from time to time (4 times) to extract the hydrogen peroxide (H₂O₂). The extract was filtered with a hydrophilic polyethersulfone filter unit (Millex-GP, 0.22 μm) and the quantity of H₂O₂ determined spectrophotometrically at 351 nm.

An H₂O₂ release study was performed with 100 mg of each of the CP-loaded microparticles in a release medium (Phosphate Buffered Saline (PBS), pH 7.4 at 37° C.) designed to mimic the release of hydrogen peroxide from the particles when contacting the teeth. The microparticles were dispersed in 20 mL of the release medium, placed in an orbital shaker at 150 rpm. 1 mL of aliquots were withdrawn at predetermined times, suitably diluted with the release medium, and the amount of H₂O₂ quantified spectrophotometrically at 351 nm. The withdrawn volume was immediately replaced with an equivalent volume of the fresh medium maintained at the same temperature.

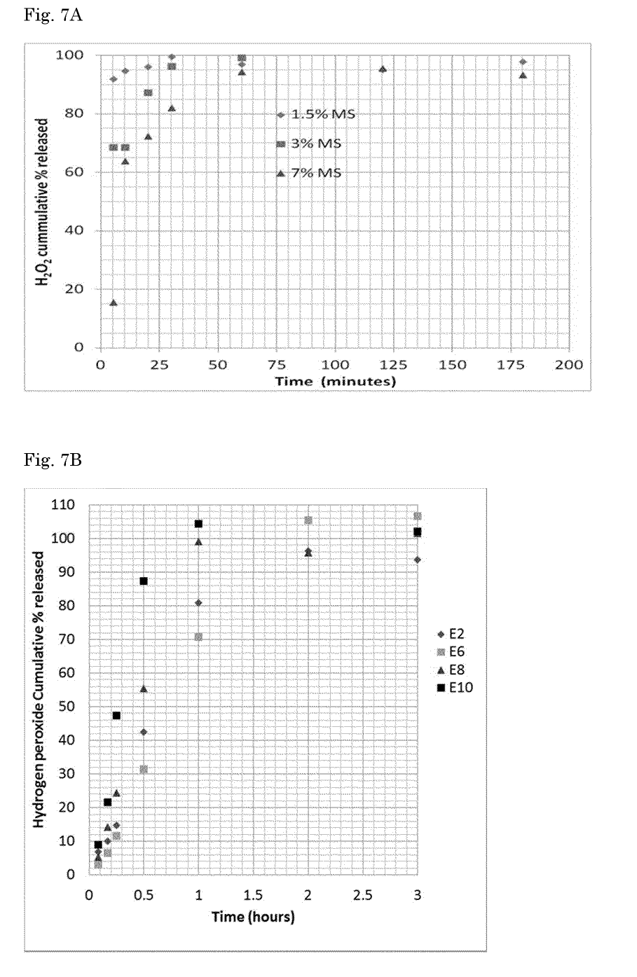

FIG. 7 shows, in 7A and 7B, a comparison of the release profiles of Eudragit microparticles prepared by O/O emulsion using acetone (A) and ethyl formate as solvent (B). Samples from FIG. 7A were formulated with 2%, 4% and 9% w/v MS in acetone for 1.5% MS, 3% MS and 7% MS w/w of formulation, respectively. Those from FIG. 7B were formulated with 4% w/v MS in ethyl formate.

TABLE 4
Composition of different formulations (sample numbers #)
of CP-loaded microparticles with Eudragit RS as a carrier.

| | % | | | % w/v | % v/v | | |
|---|---|---|---|---|---|---|---|
| | w/w | % w/v | % w/w | Surfactant | Ethyl | % w/v | |
| # | CP | MS | Precirol | in oil | formate | RS | EE (%) |
| E2 | 29.7 | 4 | 0 | 0.5 | 30 | 20 | 98 ± 1 |
| E6 | 29.4 | 7 | 0 | 0.5 | 40 | 35 | 94 ± 3 |
| E8 | 29.4 | 0 | 7 | 0.5 | 28.6 | 35 | 94 ± 2 |
| E10 | 28.6 | 0 | 5 | 0.5 | 40 | 20 | 82 ± 3 |
| A1 | 29.5 | 2 | 0 | 0 | — | 20 | 63 ± 2 |
| A2 | 29.6 | 4 | 0 | 0 | — | 20 | 67 ± 4 |
| A3 | 29.4 | 9 | 0 | 0 | — | 20 | 78 ± 2 |

TABLE 5
example of stability study of Eudragit RS microparticles prepared
by O/O emulsion using acetone (A) and ethyl formate as solvent
(E2 & E6) stored in the fridge at 5° C.

| | H₂O₂ (g/100 of sample) | | |
|---|---|---|---|
| Time (day) | E2 | E6 | A1 |
| 1 | 9.1 ± 0.7 | 8.7 ± 0.5 | 5.8 ± 1 |
| 20 | 9.3 ± 0.5 | 8.9 ± 0.3 | 4.8 ± 0.6 |
| 30 | 8.5 ± 1 | 8.5 ± 0.8 | No detection of |
| | | | H₂O₂ |

### EXAMPLE 8

Carbamide peroxide (CP) loaded-microparticles were made via emulsion solvent evaporation using ethyl formate as solvent and CAB as a carrier as described in Example 3 according to the formulations (sample numbers #) presented in Table 6.

TABLE 6
(Example 8) Composition of CAB
microparticles loaded with CP-loaded.

| | | | | % v/v | | |
|---|---|---|---|---|---|---|
| | | % w/v | % w/v | Ethyl | % w/v | |
| # | % w/w CP | MS | PGPR | formate | CAB | EE (%) |
| E32 | 29.3 | 4 | 0.5 | 30 | 20 | 89. ± 4 |
| E41 | 27.8 | 6 | 0.5 | 30 | 20 | 91. ± 6 |

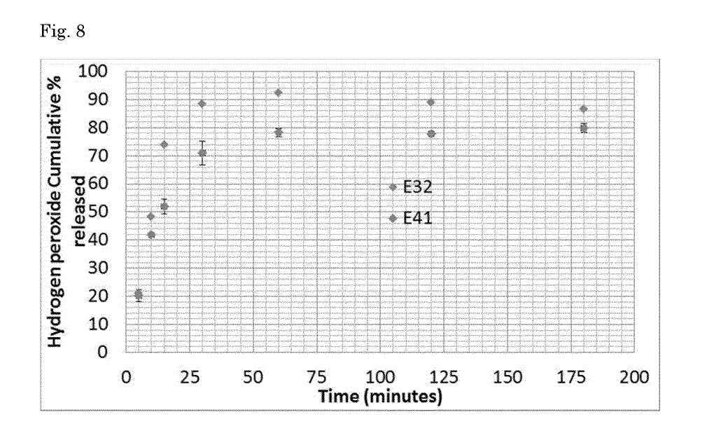

FIG. 8 is a graph showing the release profile of hydrogen peroxide from CAB microparticles loaded-CP.

1. A process for encapsulating a water-soluble oral care active agent by a shell comprising a hydrophobic polymer, the process comprising (a) dissolving the polymer in a solvent selected from the group consisting of ethyl formate, ethyl acetate, methyl acetate, propyl acetate, butyl acetate, isobutylacetate, isopropyl acetate, isoamyl acetate, and mixtures thereof, so as to form a polymer solution; (b) dispersing or dissolving the oral care active agent in the polymer solution; (c) combining the polymer solution with an oily liquid so as to provide a system comprising the polymer solution and the oily liquid; raising temperature so as to extract solvent from the solution, and evaporating the solvent thereby allowing polymer particles to solidify.

2. A process according to claim 1, wherein the step of combining the polymer solution with the oily liquid is conducted as an emulsifying step, so as to form an emulsion of the polymer solution in the oily liquid.

3. A process according to claim 1, wherein the step of combining the polymer solution with the oily liquid is conducted such that the oily liquid is allowed to act as an anti-solvent for the polymer, so as to bring about particle precipitation.

4. A process according to claim 1,
wherein the temperature is raised to a temperature in a range of from 10° C. to 35° C.

5. A process according to claim 1, wherein the polymer is selected from the group consisting of cellulose acetate butyrate, copolymers of ethyl acetate and methyl methacrylate, poly(methyl methacrylate), and mixtures thereof.

6. A process according to claim 1, wherein the oily liquid is selected from the group consisting of paraffin oil, mineral oil, heptane, dodecane, hexadecane, methyl myristate, methyl laurate, and mixtures thereof.

7. A process according to claim 1, wherein the solvent is ethyl formate.

8. A process according to claim 1, wherein the oily liquid is paraffin oil.

9. A process according to claim 1, wherein the oral care active agent is selected from the group consisting of whitening agents, antiplaque agents, anti-tartar agents, anti-gingivitis agents, anti-bacterial agents, and combinations thereof.

10. A process according to claim 9, wherein the oral care active agent is carbamide peroxide.

11. A process according to claim 9, wherein the oral care active agent is chlorhexidine.

12. A microparticle comprising a water-soluble oral care active agent encapsulated by a shell, the microparticle being obtainable by a process according to claim 1, said being coated with polyisobutylene.

13. (canceled)

14. The use of a solvent selected from the group consisting of ethyl formate, ethyl acetate, methyl acetate, propyl acetate, butyl acetate, isobutylacetate, isopropyl acetate, isoamyl acetate, and mixtures thereof, as a solvent for a hydrophobic polymer in making capsules comprising a water-soluble oral care active agent encapsulated in a shell of the polymer.

15. A use according to claim 14, wherein the method of making the capsules is a process.
